(19) United States
(12) Patent Application Publication
(10) Pub. No.: US 2025/0245798 A1
(43) Pub. Date: Jul. 31, 2025

(54) UTILIZING A LEARNING MACHINE FOR CAUSE-AND-EFFECT TESTING AND FORMULATION OF PHASE SEPARATION CHEMISTRY

(71) Applicant: Halliburton Energy Services, Inc., Houston, TX (US)

(72) Inventors: Mateusz Michal Dyngosz, Houston, TX (US); Alejandra Inmaculada Lopez Trosell, Houston, TX (US); Bo Liang, Houston, TX (US)

(21) Appl. No.: 18/422,950

(22) Filed: Jan. 25, 2024

(51) Int. Cl. G06T 7/00; G01N 33/28; G06T 7/62

(52) U.S. Cl. CPC G06T 7/0002; G01N 33/2823; G06T 7/62; G06T 2207/10016; G06T 2207/20081

(57) ABSTRACT

A method comprises obtaining a fluid sample produced from a subsurface formation. The method comprises loading the fluid sample into a device configured with one or more cameras. The method comprises obtaining, via the one or more cameras, media content of the fluid sample. The method comprises determining, via a learning machine, one or more sample properties of the fluid sample based on the media content.

### FIELD

Some implementations relate generally to the field of fluid analysis and more particularly to the field of determining properties of a fluid produced from a subsurface formation.

### BACKGROUND

In hydrocarbon recovery operations, one or more fluids (such as oil, water, gas, etc.) may be produced from a reservoir within the Earth's subsurface. Emulsion may often occur during the production, transportation, and processing of the fluids. The emulsification process may be a natural phenomenon that may result from the interaction of oil, water, and various impurities present in the reservoir fluids. Techniques, such as utilizing chemical compositions, may be employed to break the emulsion and facilitate the separation of the fluid phases.

### BRIEF DESCRIPTION OF THE DRAWINGS

Implementation of the disclosure may be better understood by referencing the accompanying drawings.

FIG. 1 is a diagrammatic illustration of an example well system, according to some implementations.

FIG. 2 is an illustration of an example device, according to some implementations.

FIG. 3 is a flowchart of example operations for determining sample properties of a fluid sample, according to some implementations.

FIG. 4 is a flowchart depicting example operations to configure a learning machine, according to some implementations.

FIG. 5 is a flowchart depicting example operations to train a learning machine, according to some implementations.

FIG. 6 is a block diagram depicting an example computer, according to some implementations.

### DESCRIPTION

The description that follows includes example systems, methods, techniques, and program flows that embody aspects of the disclosure. However, it is understood that this disclosure may be practiced without these specific details. For instance, this disclosure refers to one or more cameras disposed in a device to capture media content of a fluid sample for input into a convolutional neural network to determine one or more sample properties. Aspects of this disclosure can also be applied to any other configuration of a device to obtain inputs for any suitable learning machine to determine properties of the fluid. For clarity, some well-known instruction instances, protocols, structures, and techniques have been omitted.

Example implementations relate to obtaining data in changes in emulsion tendency as a function of surfactancy changes in the physical properties of an emulsion sample (i.e., fluid sample) including chemical moieties of effective phase separation chemicals. The fluid sample may include one or more phases of fluid (such as oil, water and/or gas), where emulsion may occur between the phases. For example, the fluid sample may include oil-in-water emulsion, water-in-oil emulsion, gas-in-oil, gas-in-water, and complex emulsions. In some implementations, phase separation chemicals (such as emulsion breakers) may be applied to the fluid sample to demulsify the phases. Sample properties of a fluid sample such as water drop volume, water quality, interface quality, etc. may be measured that may indicate the effectiveness of the phase separation chemicals. Conventional methods for classification of the sample properties may include human expertise, experience, and visual examination. The conventional methods may suffer from poor repeatability and may be subject to preferences and abilities of one to recognize certain “dirtiness” of water, interface quality, etc. after breaking emulsion. This may present challenges in consistently providing quality information and the same information in regard to the fluid sample and phase separation chemical. In some implementations, subject matter experts may be consulted to improve consistency. However, the process may be time consuming and may also impact the how the picture of the fluid sample may be taken and/or by whom.

In some implementations, a learning machine may be utilized to determine one or more sample properties of a fluid sample. The learning machine may be trained to determine sample properties such as water drop volume, water quality, interface quality, etc. of a fluid sample to assist in the testing of phase separation chemicals on a fluid sample. In some implementations, the learning machine may be based on convolutional neural network (CNN). CNNs may be capable of detecting changes in media content (such as images, videos, etc.) and may be useful for tasks such as object detection, picture classification, etc. In some implementations, a device may be configured with one or more cameras. A fluid sample (such as a fluid sample of oil and/or water and/or gas produced from a subsurface formation) may be obtained and loaded into a holder (such as a transparent bottle). The holder, with the fluid sample, may be loaded into the device and media content of the fluid sample may be obtained via the one or more cameras. The learning machine may then be employed to determine one or more properties of the fluid sample based on the media content. In some implementations, the sample properties may indicate the effectiveness of the phase separation chemical applied to the fluid sample. For example, the images captured over a period of time may indicate an increase in water quality and interface quality due to the phase separation chemical. Determining sample properties via the learning machine may reduce any issues with the overall problems stemming from bias that human based assessment of the fluid sample may suffer. In some implementations, it may also run analysis of the fluid sample faster and/or more frequently than human based assessment.

In some implementations, operations may be performed based on the sample properties. For example, the sample properties may be utilized in analyzing the effectiveness of the phase separation chemical on the fluid sample. Accordingly, operations may be performed. For instance, the chemical composition of the phase separation chemical may be adjusted, redesigned, reformulated, follow up testing on the fluid sample may be performed, etc.

### Example Systems

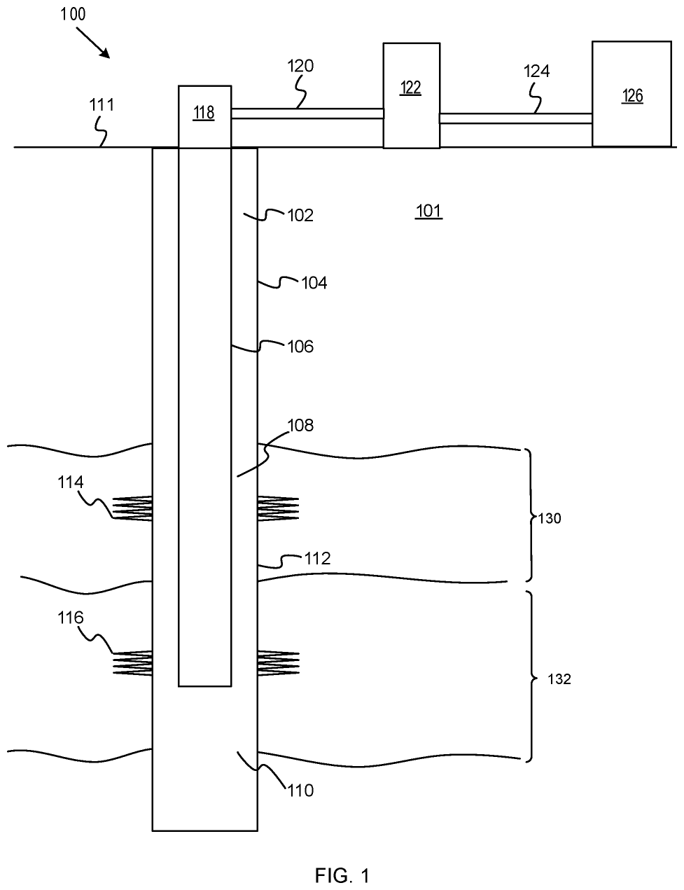

FIG. 1 is a diagrammatic illustration of an example well system, according to some implementations. In particular, a well system 100 of FIG. 1 includes a wellbore 102 in a subsurface formation 101. The wellbore 102 includes casing 104 and number of perforations 114, 116 being made in the casing 104. Each set of perforations 114, 116 is located in a respective reservoir 130, 132 to allow reservoir fluids (i.e., oil, water, and gas) from the respective reservoirs 130, 132 to flow into the wellbore 102 and into the tubular string 106 (the production tubing).

A flowline 120 coupled to the wellhead 118 of wellbore 102 and a separator 122 may allow the fluid produced up the tubular string 106 to flow to the separator 122. The separator 22 may be designed to separate the phases of the fluid produced from the wellbore 102. For instance, oil, water, and gas may be separated from each other after passing through the separator 122. The aggregate of fluid produced from wellbore 102 may then flow to a tank battery, via flowline 124, that may include components such as storage tank 126, to store the different phases of fluid in respective tanks. In some implementations, emulsion may occur within the fluid. For example, the produced oil within the oil storage tank may include water, the produced water within the water storage may include oil, etc. In some implementations, phase separation chemical may be applied to fluid phases to break the emulsion, resulting in optimizing the recovery of hydrocarbons, increasing the quality of the produced fluids, etc. For example, the phase separation chemical may be applied down the wellbore 102, in the flowline 120, in the separator 122, in the storage tanks such as storage tank 126, downstream of the well system (such as fluid transportation infrastructure), etc. Alternatively, or in addition to, samples of fluid may be obtained from the well system 100 for phase separation chemical testing prior to implementing phase separation chemicals into the well system 100.

### Example Device

Examples of a device configured to capture media content of a fluid sample are now described.

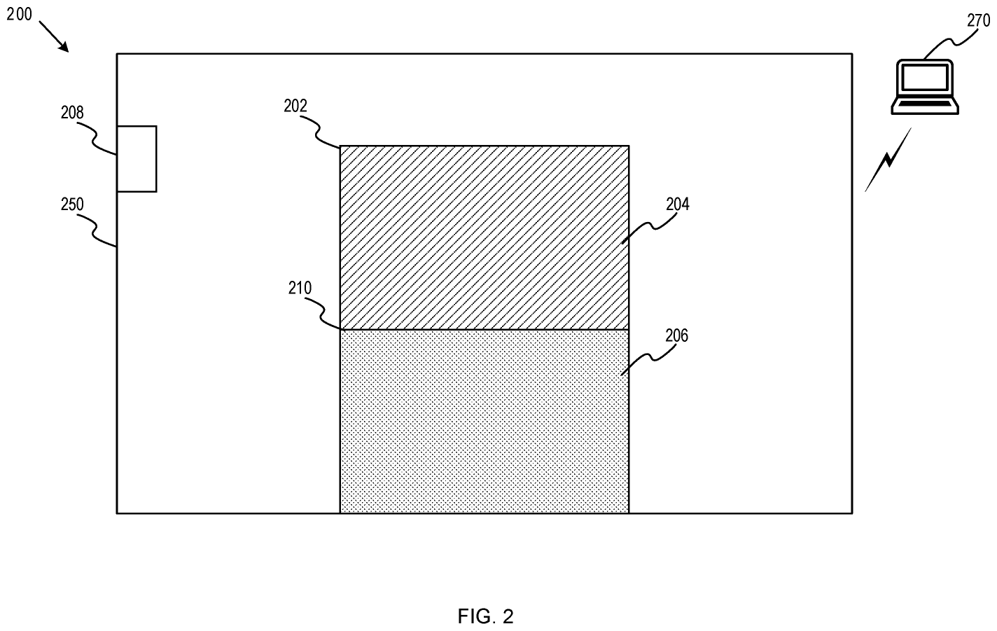

FIG. 2 is an illustration of an example device, according to some implementations. In particular, FIG. 2 depicts a cross sectional view of an example device 200. The device 200 may be configured to obtain media content of a fluid sample for determining sample properties of the fluid sample. For example, the device 200 may be communicatively coupled to a computer 270 configured to determine sample properties of the fluid sample based on the media content. In some implementations, the fluid sample may include one or more phases of fluid (such as oil, water, etc.) and phase separation chemicals added to the fluid sample. The device 200 described herein is only one example configuration of a device that may be utilized for determining sample properties of a fluid sample. Any suitable device configured with one or more cameras may be used to obtain media content of a fluid sample for determining sample properties for phase separation chemical testing. For example, components may include one or more cameras, a container where the fluid sample within the container is visible, a component where the container may be positioned on/in (such as a stand), etc.

The device 200 may include a holder 202. The holder 202 may be standardized such that it may be compatible with the device 200 (such as a bottle, beaker, etc.). In some implementations, the holder 202 may be configured such that the fluid sample is visible through the holder 202. For example, the holder 202 may be of a transparent material such as a glass, plastic, etc. The fluid sample within the holder 202 may include more than one phase. For example, FIG. 2 depicts a fluid sample with two phases (oil 204 and water 206), with a fluid interface 210 between the two phases. The holder 202, comprising the fluid sample, may be loaded into the chamber 250 of the device 200. In some implementations, chamber 250 may be configured to handle multiple holders 202, each with a fluid sample comprising one or more phases of fluid. In some implementations, a phase separation chemical may be applied to the fluid sample in the holder 202.

The device 200 may include one or more cameras such as camera 208. FIG. 2 depicts one camera 208, but implementations may have more than one camera 208, e.g., two cameras, three cameras, or more. Each camera 208 may be oriented towards the holder 202 to capture media content (one or more pictures, videos, etc.) of the fluid sample within the holder. For example, the camera 208 may obtain one or more pictures of the fluid sample at specified time intervals (i.e., 30 seconds, 5 minutes, 1 hour, etc.) for a period of time, record one or more videos of the fluid sample for a period of time, etc. Media content of the fluid sample may be captured over a period of time to capture the changes in sample properties of the fluid sample over said time period. In some implementations, the device 200 may include multiple holders 202. One camera 208 may be oriented to capture media content from all of the holders 202, each holder 202 may have a corresponding camera 208, each holder 202 may have multiple cameras 208, one or more cameras 208 may correspond to a subset of holders 202, etc.

The device 200 includes a computer 270 that may be communicatively coupled to other parts of the device 200, such as camera 208. The computer 270 may be local or remote to the device 200. In some implementations, the processor of the computer 270 may be employed to run a learning machine for performing operations to determine sample properties of the fluid sample in the holder 202 based on the media content of the fluid sample obtained by the camera 208 (as further described below). The learning machine may be trained on media content of different fluid samples to ensure its capability to recognize the different categories of clearness of water (water quality), categories of quality of interface (interface quality), water drop volume, etc. In some implementations, the computer 270 may be configured to store the sample properties in a database and/or to remote storage (i.e., the cloud). In some implementations, the sample properties may be displayed on a graphic user interface of the computer 270 or any remote device for further analysis.

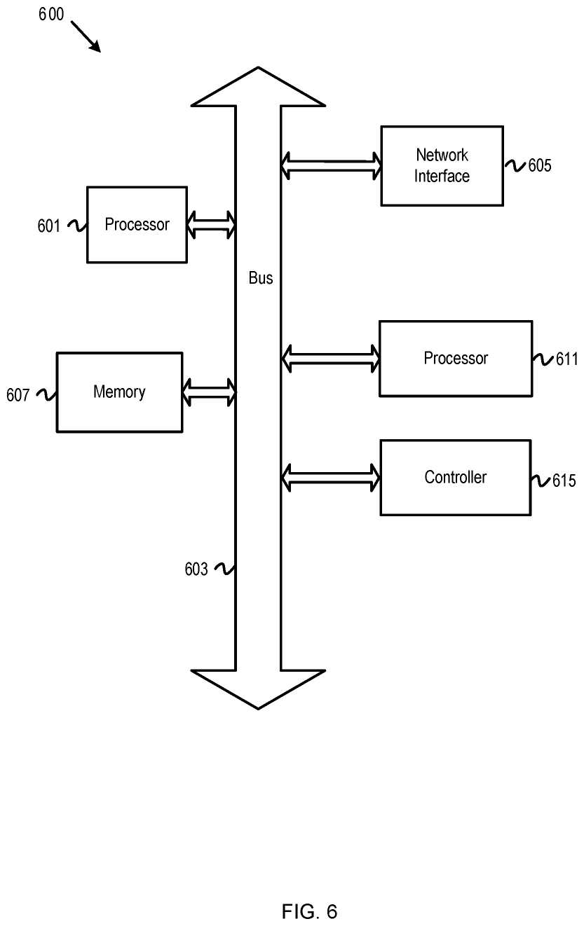

In some implementations, the processor of the computer 270 may control operations of the device 200 or subsequent operations. For instance, the processor of the computer 270 may control the camera 208 to obtain media content of the fluid sample in the holder 202. The processor may then perform operations based on the sample properties determined by the learning machine, such as adjusting the chemical composition of the phase separation chemical applied to the fluid sample. An example of the computer 270 is depicted in FIG. 6, which is further described below.

### Example Operations

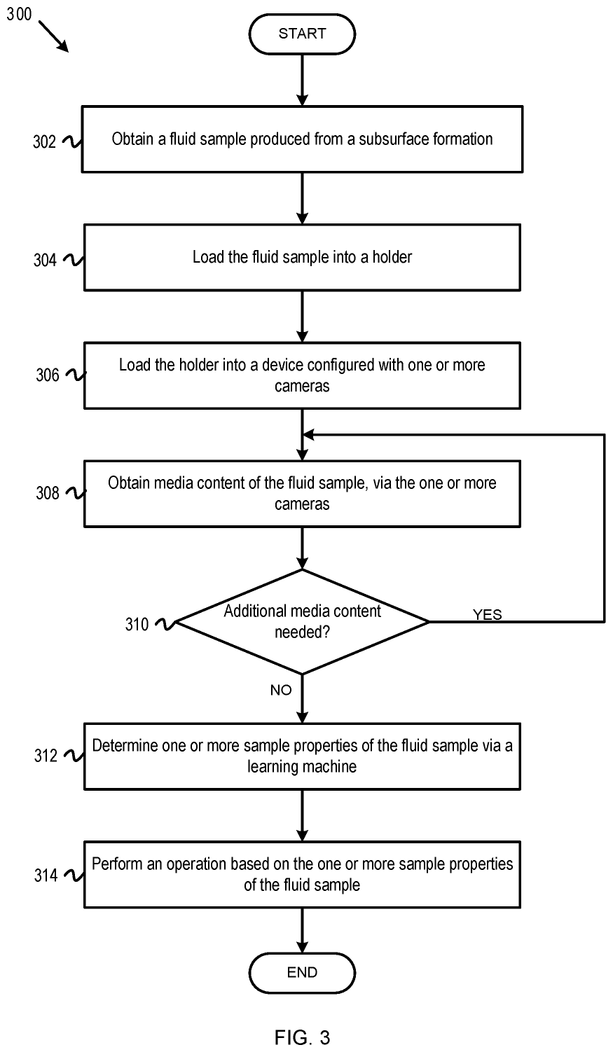

FIG. 3 is a flowchart of example operations for determining sample properties of a fluid sample, according to some implementations. FIG. 3 depicts a flowchart 300 of operations to determine sample properties of a fluid sample, via a learning machine, for phase separation chemical testing. The operations of flowchart 300 are described in reference to the well system 100 of FIG. 1 and the computer 270 of FIG. 2. Additionally, the device described in the operations of the flowchart 300 is described in reference to the device 200 of FIG. 2. Operations of the flowchart 300 begin at block 302.

At block 302, a fluid sample produced from a subsurface formation may be obtained. The fluid sample may be obtained from sources such as equipment from the well system 100, downstream of a well system 100, etc. The fluid sample may include one or more phases of fluid such as oil, water, and gas and any contaminates originating from the reservoir and/or other equipment the fluid may interact with. In some implementations, the fluid sample may include emulsion such as water-in-oil emulsion, oil-in-water emulsion, etc.

At block 304, the fluid sample may be loaded into a holder. For example, the fluid sample may be loaded into a holder such as holder 202 of FIG. 2. The fluid sample may be visible through the holder such that properties of the fluid such as water quality, interface quality, water drop volume, etc. are visible through the holder.

In some implementations, a phase separation chemistry may be applied to the fluid sample in the holder. The phase separation chemical may include any chemicals suitable for stabilizing or destabilizing emulsions such as emulsifying agents and/or surfactants. These may include molecules that have both hydrophilic (water-attracting) and hydrophobic (oil-attracting) portions. The hydrophilic part of the surfactant may associate with water molecules, while the hydrophobic part may associate with oil molecules in an attempt to stabilize or destabilize the interface between the different phases. In some implementations, the phase separation chemical may be added to the fluid sample prior to the fluid sample being loaded into the holder.

At block 306, the holder may be loaded into a device configured with one or more cameras. Each camera, such as the camera 208 of FIG. 2, may have the ability to capture media content (such as one or more pictures, one or more videos, etc.) of the fluid sample in the holder.

At block 308, the processor of the computer may obtain media content of the fluid sample, via the one or more cameras. For example, an image of the fluid sample may be obtained by a camera. In some instances, a video of the fluid sample over a period of time (e.g., 1 hour, 24 hours, 3 days, etc.) may be obtained.

At block 310, the processor of the computer 270 may determine if additional media content is needed. If additional media content of the fluid sample is needed, then operations return to block 308 to obtain additional media content. For example, to evaluate the phase separation chemistry, images of the fluid sample over a period of time may be required. Thus, operations may return to block 308 to capture another image of the fluid sample at the next time interval. For instance, images of the fluid sample may be obtained every hour for a total time period of 24 hours such that a learning machine may evaluate the fluid sample (as described below). In some implementations, the media content first obtained in block 308 may be the fluid sample with no phase separation chemical. Prior to returning to block 308 to obtain additional media content, a phase separation chemical may be added to the fluid sample such that the learning machine may utilize the first media content as a comparison to the subsequent media content. If no additional media content is needed, then operations of the flowchart 300 proceed to block 312.

At block 312, the processor of the computer 270 may determine one or more sample properties of the fluid sample via a learning machine. The sample properties may include water quality, interface quality, water drop volume, etc. The water quality may indicate how clear/clean the water is. For example, poor water quality may indicate there are oil droplets in the water. In some implementations, the water quality may be measured on a scale, such as 1 through 5, where 1 represents a poor water quality and a 5 represents a good water quality. Any suitable units and/or scale may be utilized to indicate the water quality. Similarly, the interface quality may be measured utilizing any suitable units and/or scale to indicate the interface quality between the different phases within the fluid sample. The interface quality may indicate how clean/precise the interface between phases is. For example, a poor interface quality may indicate there is interfacial tension between the water drops and oil drops, resulting in a mixture of the phases. The water drop volume may be the volume of water that may drop out of the oil phase in the fluid (i.e., milliliters (mL) of water). In some implementations, the water drop volume may be obtained over a time period to determine the water drop volume rate.

The learning machine may be trained (as described below) to determine the one or more sample properties based on the media content of the fluid sample obtained in blocks 306-308. The learning machine may utilize media content to determine the sample properties. For example, one image, multiple images, a video, multiple videos, a combination of images and videos, etc. may be utilized by the learning machine. In some implementations, the learning machine may generate sample properties for each media content. For example, if 10 images that may be obtained over a period of time are input into the learning machine, the learning machine may generate sample properties for each of the images. Accordingly, a trend of the sample properties may be observed for the fluid sample over a period of time. The fluid property trend may indicate the effectiveness of the phase separation chemistry on the fluid sample over said time period.

In some implementations, the sample properties may be labeled with the phase separation chemical. For example, the media content of a fluid sample with a type of phase separation chemistry applied may be labeled with said phase separation chemical. Accordingly, the sample properties generated by the learning machine may label each of the sample properties with the type of phase separation chemistry. If a different type of phase separation chemistry is added to the fluid sample, the data of the sample properties of the corresponding phase separation chemistry may then be compared for analysis and used to direct synthesis of more effective separation chemicals.

At block 314, the processor of the computer 270 may perform an operation based on the sample properties of the fluid sample. For example, operations such as further testing on the sample, adjustment, redesign, or reformulation of the phase separation chemistry may be made, etc. For instance, the sample properties may indicate a good water quality but a poor interface quality in the fluid sample. Accordingly, the composition of the phase separation chemistry may be adjusted, redesigned, or reformulated to improve the interface quality and maintain the good water quality. In some implementations, the sample properties of a fluid sample may be displayed (i.e., on the graphic user interface of the computer 270 or any other suitable device, software, etc.) such that the sample properties may be analyzed. For example, the trend of the sample properties over a period of time may be analyzed to determine if the composition of the phase separation chemistry may need to be adjusted, redesigned, or reformulated. Alternatively, or in addition to, sample properties from two different tests (i.e., blocks 302-312 were repeated with a fluid sample with different phase separation chemistry) may be displayed to compare phase separation chemical effectiveness (i.e., which phase separation chemical results in the preferred water quality, interface quality, water drop volume rate, etc.).

Selection of the chemistries for the phase separation chemical maybe made by virtue of measurements of the molecules such as relative solubility number (RSN) number, hydroxyl number, hydrophilic-lipophilic balance (HLB), calculated structure or any other method of measuring molecular conformance. The measurements and any other data related to the fluid sample and/or phase separation chemistry (such as chemical moieties of the phase separation chemical) may be compared to the sample properties generated by the learning machine to direct synthesis of more effective separation chemicals. For example, 3 phase separation chemicals with molecules of surfactants may have a similar head group but different tails. For instance, a first chemical includes 10 ethoxylated groups, a second chemical includes 20 ethoxylated groups, and a third chemical includes 30 ethoxylated groups. The three phase separation chemicals may be applied to a fluid sample and the phase properties may be determined, via the learning machine, for each respective phase separation chemical. The respective phase properties may indicate the first chemical is not good (i.e., water quality, interface quality, water drop volume, etc. is not satisfactory), the second chemical is better than the first chemical, and the third chemical is better than the second chemical. Due to the increasing trend in performance relative to the increase in ethoxylated groups, a fourth chemical with more than 30 ethoxylated groups may be synthesized to determine if the trend may continue such that a more effective phase separation chemical may be supplied. This method of synthesizing may be conducted when changing any other suitable phase chemical properties such as the groups/moieties, conformation of the molecule, etc.

In some implementations, the operations of the flowchart 300 may be repeated for each fluid property or group of sample properties. For example, the operations of the flowchart 300 may be performed to identify a chemistry for the phase separation chemicals that returns a satisfactory water drop volume. The operations of the flowchart 300 may then subsequently be performed to identify a chemistry for the phase separation chemicals that returns a satisfactory water quality and then a satisfactory interface quality. Alternatively, the operations of the flowchart 300 may be utilized for determining a chemistry for both water quality and interface quality. The operations of the flowchart 300 may then subsequently be performed to determine the chemistry that may return a satisfactory water drop volume. The repeatability of the flowchart 300 may be performed in any suitable order and/or for determining phase separation chemistries associated with any combination of sample properties.

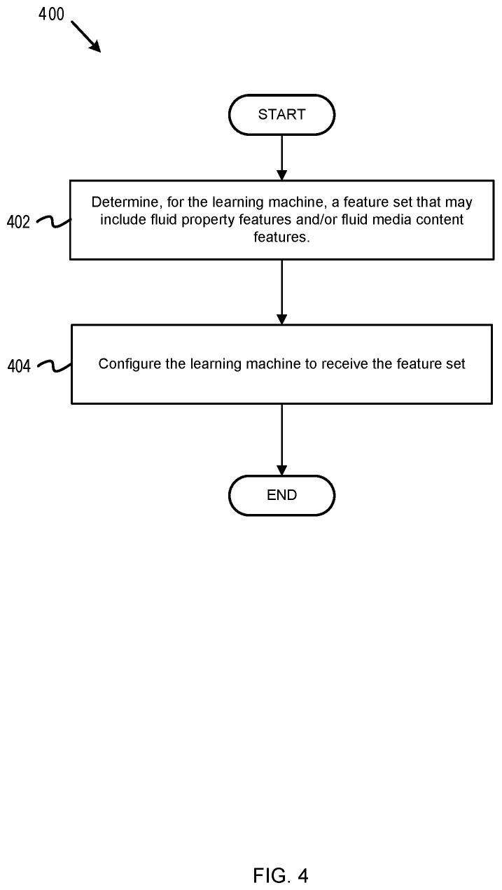

FIG. 4 is a flowchart depicting example operations to configure a learning machine, according to some implementations. FIG. 4 includes a flowchart 400 that may determine a feature set, and may configure the learning machine to receive the feature set as input. The learning machine may include any suitable learning machine such as a convolutional neural network (CNN). Operations of flowchart 400 of FIG. 4 are described in reference to the processor of the computer 270 of FIG. 2. Operations of the flowchart 400 start at block 402.

At block 402, the processor of the computer 270 may determine, for the learning machine, a feature set that may include fluid property features and/or fluid media content features. A fluid property feature may include features associated with the properties of a fluid sample prior to, during, and/or after phase separation chemicals are applied to the fluid sample such as water drop volume, water quality, interface quality, etc. A fluid media content feature may include features associated with images, videos, etc. of a fluid sample. Some implementations may utilize any suitable feature set including any suitable value related to the media content of the fluid samples utilized in testing phase separation chemical(s).

At block 404, the processor of the computer 270 may configure the learning machine to receive the feature set as input. As noted, the features may include a fluid property feature and/or a fluid media content feature. The flowchart 400 ends after block 404.

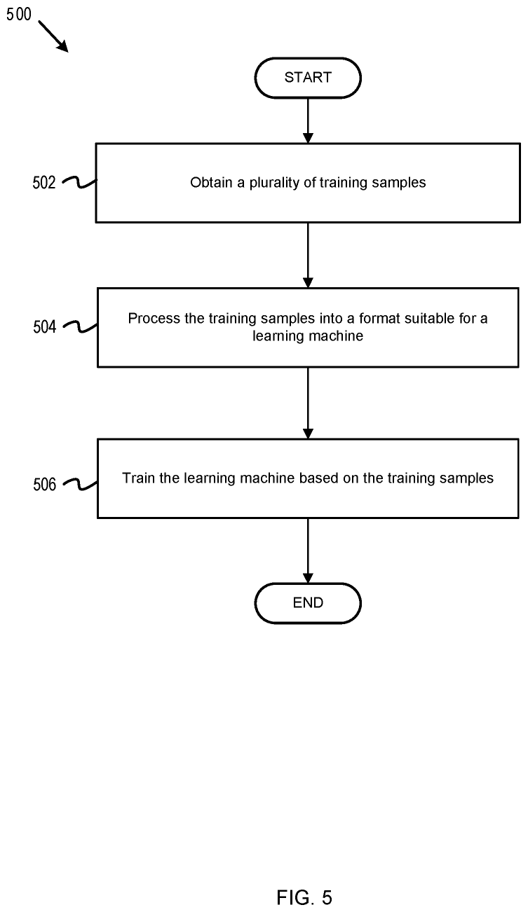

After block 404, the learning machine may begin training itself based on training samples. The discussion of FIG. 5 provides additional details about training samples and training the learning machine.

FIG. 5 is a flowchart depicting example operations to train a learning machine, according to some implementations. FIG. 5 includes a flowchart 500 that may train a supervised and/or unsupervised learning machine with training samples. Operations of flowchart 500 of FIG. 5 are described in reference to the processor of the computer 270 of FIG. 2. Operations of the flowchart 500 start at block 502.

At block 502, the processor of the computer 270 may obtain a plurality of training samples. The training samples may ensure the learning machine's capability to recognize different sample properties. Each training sample may be associated with a fluid sample. The training samples may include one or more fluid property samples and one or more fluid media content samples. The fluid property sample and fluid media content samples may be labeled with the fluid phase types, phase separation chemistry time, etc. For example, an image of a fluid sample comprising oil and water may be obtained. Additionally, the fluid sample may include a phase separation chemical. Sample properties may be obtained (such as through lab testing of samples, visual analysis of the fluid sample, etc.). The sample properties may be utilized as a fluid property sample and the image of the fluid sample may be utilized as a fluid media content sample for training the learning machine, where each of the samples may be labeled with phase separation chemicals added to the fluid sample, time phase separation chemicals have been in the fluid sample, etc. The training samples may be generated by software and systems based on the system level design, numerical modeling, sample measurements, etc. For example, synthetic data may be generated and may be the labeled with fluid types, phase separation chemistries, time, etc. to generate training samples. Some implementations may utilize any suitable technique to obtain training samples.

At block 504, the processor of the computer 270 may process the training samples into a format suitable for a learning machine. For instance, if the learning machine is configured to accept inputs with values between 0 and 1, the fluid property sample may be scaled to values between 0 and 1.

At block 506, the processor of the computer 270 may train the learning machine based on the training samples. The learning machine may use fewer than all the training samples in its training process. For example, the learning machine may utilize 80% of the training samples at block 504. Later, the learning machine may use the remaining 20% of the training samples to test the learning machine. The learning machine may be updated (i.e., trained) as new training samples are obtained. For instance, the learning machine be trained with updated training samples obtained from synthetic data, historical testing data, etc.

While the aspects of the disclosure are described with reference to various implementations and exploitations, it will be understood that these aspects are illustrative and that the scope of the claims is not limited to them. In general, determining sample properties via a learning machine for testing phase separation chemistries as described herein may be implemented with facilities consistent with any hardware system or hardware systems. Many variations, modifications, additions, and improvements are possible.

Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the disclosure. In general, structures and functionality presented as separate components in the example configurations may be implemented as a combined structure or component. Similarly, structures and functionality presented as a single component may be implemented as separate components. These and other variations, modifications, additions, and improvements may fall within the scope of the disclosure.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example process in the form of a flow diagram. However, some operations may be omitted and/or other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described should not be understood as requiring such separation in all implementations, and the described program components and systems may generally be integrated together in a single software product or packaged into multiple software products. Additionally, other implementations are within the scope of the following claims. In some cases, the actions recited in the claims may be performed in a different order and still achieve desirable results.

### Example Computer

FIG. 6 is a block diagram depicting an example computer, according to some implementations. FIG. 6 depicts a computer 600 for determining sample properties of a fluid sample. The computer 600 includes a processor 601 (possibly including multiple processors, multiple cores, multiple nodes, and/or implementing multi-threading, etc.). The computer 600 includes memory 607. The memory 607 may be system memory or any one or more of the above already described possible realizations of machine-readable media. The computer 600 also includes a bus 603 and a network interface 605. The computer 600 can communicate via transmissions to and/or from remote devices via the network interface 605 in accordance with a network protocol corresponding to the type of network interface, whether wired or wireless and depending upon the carrying medium. In addition, a communication or transmission can involve other layers of a communication protocol and or communication protocol suites (e.g., transmission control protocol, Internet Protocol, user datagram protocol, virtual private network protocols, etc.).

The computer 600 also includes a processor 611 and a controller 615 which may perform the operations described herein. For example, the processor 611 may obtain media content of a fluid sample and determine one or more properties of the fluid sample to be utilized in analysis of a phase separation chemical applied to the fluid sample. The controller 615 may perform an operation based on the sample properties, such as adjusting the composition of the phase separation chemical for further testing. The processor 611 and the controller 615 can be in communication. Any one of the previously described functionalities may be partially (or entirely) implemented in hardware and/or on the processor 601. For example, the functionality may be implemented with an application specific integrated circuit, in logic implemented in the processor 601, in a co-processor on a peripheral device or card, etc. Further, realizations may include fewer or additional components not illustrated in FIG. 6 (e.g., video cards, audio cards, additional network interfaces, peripheral devices, etc.). The processor 601 and the network interface 605 are coupled to the bus 603. Although illustrated as being coupled to the bus 603, the memory 607 may be coupled to the processor 601.

### EXAMPLE IMPLEMENTATIONS

Implementation #1: A method comprising: obtaining a fluid sample produced from a subsurface formation; loading the fluid sample into a device configured with one or more cameras; obtaining, via the one or more cameras, media content of the fluid sample; and determining, via a learning machine, one or more sample properties of the fluid sample based on the media content.

Implementation #2: The method of Implementation #1, wherein the one or more sample properties of the fluid sample comprises a property selected from the group consisting of water drop volume, water quality, interface quality, and any combination thereof.

Implementation #3: The method of Implementation #1 or #2, wherein the media content of the fluid sample includes one or more pictures of the fluid sample from the respective cameras and one or more videos of the fluid sample from the respective cameras.

Implementation #4: The method of any one or more of Implementation #1-3, wherein the fluid sample includes comprises a fluid selected from the group consisting of water, oil, gas, and any combination of fluids thereof.

Implementation #5: The method of any one or more of Implementation #1-4 further comprising: determining, for the learning machine, a feature set including a fluid property feature and a fluid media content feature; and configuring the learning machine to receive the feature set as input.

Implementation #6: The method of any one or more of Implementation #1-5 further comprising: training the learning machine to generate the one or more sample properties based on a plurality of training samples, the training samples including fluid media content samples and fluid property samples.

Implementation #7: The method of any one or more of Implementation #1-6 further comprising: adding one or more phase separation chemicals to the fluid sample; obtaining, via the one or more cameras, the media content of the fluid sample at time intervals over a time period; and determining, via the learning machine, the one or more sample properties of the fluid sample at different time intervals over the time period based on the respective media content.

Implementation #8: The method of any one or more of Implementation #1-7 further comprising: performing an operation based on the one or more sample properties of the fluid sample.

Implementation #9: The method of Implementation #8 the operation further comprising: obtaining the sample properties from the fluid sample with first phase separation chemicals; and directing synthesis of second phase separations chemicals based on the sample properties.

Implementation #10: A system comprising: a device configured with one or more cameras; a sample holder configured to hold a fluid sample produced from a subsurface formation; a processor; and a computer-readable medium having instructions stored thereon that are executable by the processor, the instructions including, instructions to obtain, via the one or more cameras, media content of the fluid sample when the sample holder is loaded into the device, and instructions to determine, via a learning machine, one or more sample properties of the fluid sample based on the media content.

Implementation #11: The system of Implementation #10, wherein the one or more sample properties of the fluid sample comprises a property selected from the group consisting of include water drop volume, water quality, interface quality and any combination thereof.

Implementation #12: The system of Implementation #10 or #11, wherein the media content of the fluid sample includes one or more pictures of the fluid sample from the respective cameras and one or more videos of the fluid sample from the respective cameras.

Implementation #13: The system of any one or more of Implementation #10-12, wherein the fluid sample comprises a fluid selected from the group consisting of includes water, oil, gas, and any combination of fluids thereof.

Implementation #14: The system of any one or more of Implementation #10-13 further comprising: instructions to determine, for the learning machine, a feature set including a fluid property feature and a fluid media content feature; and instructions to configure the learning machine to receive the feature set as input.

Implementation #15: The system of any one or more of Implementation #10-14 further comprising: instructions to train the learning machine to generate the one or more sample properties based on a plurality of training samples, the training samples including fluid media content samples and fluid property samples.

Implementation #16: A non-transitory, computer-readable medium having instructions stored thereon that are executable by a processor, the instructions comprising: instructions to obtain, via one or more cameras, media content of a fluid sample produced from a subsurface formation, wherein the fluid sample is loaded into a device configured with the one or more cameras; and instructions to determine, via a learning machine, one or more sample properties of the fluid sample based on the media content.

Implementation #17: The non-transitory, computer-readable medium of Implementation #16, wherein the one or more sample properties of the fluid sample include water drop volume, water quality and interface quality.

Implementation #18: The non-transitory, computer-readable medium of Implementation #16 or #17, wherein the media content of the fluid sample includes one or more pictures of the fluid sample from the respective cameras and one or more videos of the fluid sample from the respective cameras.

Implementation #19: The non-transitory, computer-readable medium of any one or more of Implementation #16-18, wherein the fluid sample includes water, oil, or a combination of fluids.

Implementation #20: The non-transitory, computer-readable medium of any one or more of Implementation #16-19 further comprising: instructions to add one or more phase separation chemicals to the fluid sample; instructions to obtain, via the one or more cameras, the media content of the fluid sample at time intervals over a time period; and instructions to determine, via the learning machine, the one or more sample properties of the fluid sample at different time intervals over the time period based on the respective media content.

Use of the phrase “at least one of” preceding a list with the conjunction “and” should not be treated as an exclusive list and should not be construed as a list of categories with one item from each category, unless specifically stated otherwise. A clause that recites “at least one of A, B, and C” can be infringed with only one of the listed items, multiple of the listed items, and one or more of the items in the list and another item not listed.

As used herein, the term “or” is inclusive unless otherwise explicitly noted. Thus, the phrase “at least one of A, B, or C” is satisfied by any element from the set {A, B, C} or any combination thereof, including multiples of any element.

1. A method comprising:
obtaining a fluid sample produced from a subsurface formation;
loading the fluid sample into a device configured with one or more cameras;
obtaining, via the one or more cameras, media content of the fluid sample; and
determining, via a learning machine, one or more sample properties of the fluid sample based on the media content.

2. The method of claim 1, wherein the one or more sample properties of the fluid sample comprises a property selected from the group consisting of water drop volume, water quality, interface quality, and any combination thereof.

3. The method of claim 1, wherein the media content of the fluid sample includes one or more pictures of the fluid sample from the respective cameras and one or more videos of the fluid sample from the respective cameras.

4. The method of claim 1, wherein the fluid sample comprises a fluid selected from the group consisting of water, oil, gas, and any combination thereof.

5. The method of claim 1 further comprising:
determining, for the learning machine, a feature set including a fluid property feature and a fluid media content feature; and
configuring the learning machine to receive the feature set as input.

6. The method of claim 1 further comprising:
training the learning machine to generate the one or more sample properties based on a plurality of training samples, the training samples including fluid media content samples and fluid property samples.

7. The method of claim 1 further comprising:
adding one or more phase separation chemicals to the fluid sample;
obtaining, via the one or more cameras, the media content of the fluid sample at time intervals over a time period; and
determining, via the learning machine, the one or more sample properties of the fluid sample at different time intervals over the time period based on the respective media content.

8. The method of claim 1 further comprising:
performing an operation based on the one or more sample properties of the fluid sample.

9. The method of claim 8 the operation further comprising:
obtaining the sample properties from the fluid sample with first phase separation chemicals; and
directing synthesis of second phase separations chemicals based on the sample properties.

10. A system comprising:
a device configured with one or more cameras;
a sample holder configured to hold a fluid sample produced from a subsurface formation;
a processor; and
a computer-readable medium having instructions stored thereon that are executable by the processor, the instructions including,
instructions to obtain, via the one or more cameras, media content of the fluid sample when the fluid sample is loaded into the device, and
instructions to determine, via a learning machine, one or more sample properties of the fluid sample based on the media content.

11. The system of claim 10, wherein the one or more sample properties of the fluid sample water drop volume, water quality, interface quality and any combination thereof.

12. The system of claim 10, wherein the media content of the fluid sample includes one or more pictures of the fluid sample from the respective cameras and one or more videos of the fluid sample from the respective cameras.

13. The system of claim 10, wherein the fluid sample comprises a fluid selected from the group consisting of water, oil, gas, and any combination thereof.

14. The system of claim 10 further comprising:
instructions to determine, for the learning machine, a feature set including a fluid property feature and a fluid media content feature; and
instructions to configure the learning machine to receive the feature set as input.

15. The system of claim 10 further comprising:
instructions to train the learning machine to generate the one or more sample properties based on a plurality of training samples, the training samples including fluid media content samples and fluid property samples.

16. A non-transitory, computer-readable medium having instructions stored thereon that are executable by a processor, the instructions comprising:
instructions to obtain, via one or more cameras, media content of a fluid sample produced from a subsurface formation, wherein the fluid sample is loaded into a device configured with the one or more cameras; and
instructions to determine, via a learning machine, one or more sample properties of the fluid sample based on the media content.

17. The non-transitory, computer-readable medium of claim 16, wherein the one or more sample properties of the fluid sample include water drop volume, water quality and interface quality.

18. The non-transitory, computer-readable medium of claim 16, wherein the media content of the fluid sample includes one or more pictures of the fluid sample from the respective cameras and one or more videos of the fluid sample from the respective cameras.

19. The non-transitory, computer-readable medium of claim 16, wherein the fluid sample includes water, oil, or a combination of fluids.

20. The non-transitory, computer-readable medium of claim 16 further comprising:
instructions to add one or more phase separation chemicals to the fluid sample;
instructions to obtain, via the one or more cameras, the media content of the fluid sample at time intervals over a time period; and
instructions to determine, via the learning machine, the one or more sample properties of the fluid sample at different time intervals over the time period based on the respective media content.
